(19) United States
(12) Patent Application Publication
(10) Pub. No.: US 2023/0074374 A1
(43) Pub. Date: Mar. 9, 2023

(54) METHODS OF MANUFACTURING EXTRACELLULAR MATRIX USING ASPARTYL ALANYL DIKETOPIPERAZINE (DA-DKP)

(71) Applicant: PUR Biologics, Inc, Ladera Ranch, CA (US)

(72) Inventor: Francis Christopher ZEIGLER, Ladera Ranch, CA (US)

(21) Appl. No.: 17/800,134

(22) PCT Filed: Feb. 16, 2021
(86) PCT No.: PCT/US2021/018220

§ 371 Date: Aug. 16, 2022

Related U.S. Application Data
(60) Provisional application No. 62/977,377, filed on Feb. 16, 2020.

(51) Int. Cl. C12N 5/077; A61K 35/33; A61K 8/65; A61Q 19/08

(52) U.S. Cl. CPC C12N 5/0656; A61K 35/33; A61K 8/65; A61Q 19/08; C12N 2501/999

(57) ABSTRACT

The present disclosure methods, compositions and kits for the manufacture of extracellular matrix (ECM), wherein the methods compositions and kits comprise aspartyl alanyl diketopiperazine (DA-DKP).

### RELATED APPLICATIONS

This application claims priority to, and the benefit of, U.S. Provisional Application No. 62/977,377, filed Feb. 16, 2020, the contents of which are incorporated by reference herein in its entirety for all purposes.

### BACKGROUND

The extracellular (ECM) is a versatile biomaterial with many cosmetic and therapeutic uses. The majority of connective tissues comprise collagens, and to a much lesser extent (based on relative abundance by weight) other glycoproteins such as laminins, fibronectin, and glycosaminoglycans (GAGs) including hyaluronic acid, and other sulfated GAGs such as aggrecan and perlecan. However, collagens are cross-linked and require enzymatic or chemical degradation to isolate for cosmetic or therapeutic uses and manufacture into useful products for human use. Examples including the use of bovine and porcine corium after pepsin digestion or chemical modification, porcine intestinal submucosa, and human cadaver-derived tissues such as skin and bone, are all widely known in the art.

Animal sources for ECM such as porcine and bovine tissues carry the risk of unwanted immune reactions, including known allergies to bovine and porcine antigens or allergens, mostly proteins, while human cells that use animal-derived components (including most often bovine serum, bovine albumin, or porcine trypsin), or non-human plant-derived proteins (including soybean trypsin inhibitor or recombinant human albumin produced in plants which can contain residual plant proteins or polypeptides) suffer from the same risks. Accordingly, the commercial usefulness of ECM from animal sources can be limited by safety concerns and/or regulatory hurdles for validating the animal components' removal to safer residual levels needed for approval to use commercially. The human immune system is sensitive enough to react to extremely low abundance antigens, and animal and plant proteins have been demonstrated to cause unwanted immune reactions, including potentially life-threatening allergic reactions which can cause anaphylactic shock and even death in some cases. Animal-derived and plant-derived protein components, and human cells grown in animal-derived or plant-derived protein components are collectively termed “xenogeneic.” On the other hand, products manufactured without contact to these animal-derived and plant-derived protein components, and consequently do not contain such components upon final purification, are known as “xeno-free” Conventional approaches for manufacturing ECM in xenogenic media can require removal of animal-derived and/or plant-derived protein components, limiting the commercial usefulness of these methods.

Previous methods for producing human ECM have utilized allogeneic tissues from cadavers. However, such cadaver-derived ECM presents risk of disease transmission, as well as limited commercial scalability, since each donor provides limited amounts of obtainable human ECM (e.g., a 70 kg human contains less than 20% by weight human collagens or no greater than a few kgs raw ECM materials). Additionally, cadaver-derived tissues involve expensive safety testing to mitigate some risk of disease transmission and must be extensively tested for a number of disease-causing pathogens including viruses and bacteria for each single cadaverous donor.

Additionally, ECM can be used for animal feed. However, as a practical matter, conventional approaches for producing ECM using cell culture or harvest from cadavers can be cost-prohibitive on a commercial scale.

Accordingly, there is a long-felt need in the art for improved methods of producing human ECM. The present disclosure addresses these needs.

### SUMMARY

The present disclosure provides a method of manufacturing ECM, the method comprising culturing a plurality of fibroblasts in a medium comprising aspartyl-alanyl-diketopiperazine (DA-DKP) for a time period sufficient for the plurality of fibroblasts to produce extracellular matrix (ECM).

The present disclosure provides a method of manufacturing ECM, the method comprising: a) culturing a plurality of fibroblasts in a medium comprising serum; b) gradually reducing the concentration of serum in the medium over a time period such that there is a reduction in the concentration of serum; and c) culturing the plurality of fibroblasts in a medium comprising DA-DKP for a time period sufficient for the plurality of fibroblasts to produce extracellular matrix (ECM). In some aspects, steps (b) and (c) can be performed sequentially, in any order. In some aspects, steps (b) and (c) can be performed concurrently.

In some aspects, a plurality of fibroblasts can cultured in a medium comprising DA-DKP for at least about 2 weeks.

In some aspects, a plurality of fibroblasts can be cultured in a medium comprising serum for at least about 2 weeks.

In some aspects, gradually reducing the concentration of serum in the medium over a time period can comprise reducing the amount of serum in the medium over the time period such that there is at least a 95% reduction in the concentration of serum. In some aspects, the concentration of serum in the medium can be gradually reduced over a time period of at least 5 days.

In some aspects, a medium comprising serum can comprise serum at a concentration of about 0.1% to about 20% (v/v).

In some aspects, a medium comprising DA-DKP can comprise DA-DKP at a concentration of about 1 nM to about 1 μM, or about 1 μM to about 10 mM, or about 1 μM to about 1 mM, or about 100 μM to about 1 mM.

In some aspects, a plurality of fibroblasts can comprise activated fibroblasts. In some aspects, activated fibroblasts can express elevated levels of at least one of: a) Fibroblast Activation Protein (FAP); b) at least one cytokine, wherein the at least one cytokine is selected from IL-6, IL-8, TGF-β and MIP-1α; and c) at least one ECM protein, wherein the ECM protein is selected from a laminin, fibronectin, collagen type I, collagen type II, collagen type III, collagen type IV, collagen type V, collagen type VI.

In some aspects, DA-DKP can be obtained from a solution comprising serum albumin. In some aspects, serum albumin can be recombinant serum albumin. In some aspects, DA-DKP can be obtained from conditioned medium that was used to culture a plurality of mammalian cells. In some aspects, DA-DKP can be obtained from serum. In some aspects, DA-DKP can be chemically synthesized.

In some aspects, the amount of ECM that is produced using the methods of the present disclosure can be at least about 10%, or at least about 50%, or at least about 100% greater than the amount of ECM that is produced under otherwise identical conditions except for the omission of DA-DKP.

In some aspects, the amount of ECM that is produced using the methods of the present disclosure can be at least about 2 times, or at least about 5 times, or at least about 10 times greater than the amount of ECM that is produced under otherwise identical conditions except for the omission of DA-DKP.

In some aspects, ECM that is produced using the methods of the present disclosure can comprise soluble ECM, mature ECM, soluble mature ECM or any combination thereof.

In some aspects, ECM that is produced using the methods of the present disclosure can comprise triple-helical or non-reducible gamma-form fibrillary collagen, or a combination of both.

In some aspects, ECM that is produced using the methods of the present disclosure can comprise about 90% (w/w/) of COL1, and about 10% (w/w) of COL5, COL4, COL5, COL6, or any combination thereof.

In some aspects, the methods of the present disclosure can further comprise isolating the ECM. In some aspects, the isolated ECM is xeno-free.

The present disclosure provides a composition comprising the ECM produced by any one of the methods of the present disclosure. In some aspects, the composition can further comprise DA-DKP. In some aspects, the composition can further comprise a plurality of fibroblasts.

In some aspects, the compositions of the present disclosure can be used as a cosmetic. Cosmetic uses included, but are not limited to, filling facial wrinkles, reducing visible aging signs, promoting hair growth, improving appearance of skin or any combination thereof.

In some aspects, the compositions of the present disclosure can be used in the treatment and/or prevention of a disease or disorder. Disease or disorders include, but are not limited to, arthritis, cancer, an autoimmune disorder, a surgical wound, pain or any combination thereof.

The present disclosure provides a kit comprising the ECM produced by any one of the methods of the present disclosure. In some aspects, the kit can further comprise DA-DKP. In some aspects, the kit can further comprise a plurality of fibroblasts.

Any of the above aspects, or any other aspect described herein, can be combined with any other aspect described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the disclosure will be apparent from the following detailed description and claim.

### DETAILED DESCRIPTION

The present disclosure is based at least in part on the surprising and unexpected discovery that the addition of DA-DKP to the medium used to culture fibroblasts increases the yield and quality of Extracellular Matrix (ECM) that is produced by the fibroblasts. Without wishing to be bound by theory, the addition of DA-DKP activates fibroblasts, thereby increasing the amount and quality of the ECM produced by the fibroblasts, leading to enhanced cosmetic and therapeutic efficacy of the produced ECM. Previously, only non-activated fibroblasts have been used for manufacturing conditioned medium and extracellular matrix, and prior methods have used primarily bovine animal serum in standard culture media of various types, or alternatively using human blood-purified, or recombinant-derived albumins. These previous methods all used traditional fibroblasts derived from neonatal foreskin, without any immunomodulatory supplements to activate the cells, since fibroblasts are not primary immune cells and would not have been thought to respond in any beneficial manner.

The present disclosure provides compositions, methods and kits for the manufacture of extracellular matrix. Additionally, the present disclosure provides compositions and kits comprising ECM produced using the methods described herein, as well as methods of using the ECM to treat diseases and/or disorders, and methods of using the ECM in cosmetics.

Methods

The present disclosure provides a method of manufacturing ECM, the method comprising culturing a plurality of fibroblasts in a medium comprising aspartyl-alanyl-diketopiperazine (DA-DKP) for a time period sufficient for the plurality of fibroblasts to produce extracellular matrix (ECM).

In some aspects of the preceding method, the medium comprising DA-DKP can further comprise albumin. In some aspects of the preceding method, the medium can further comprise recombinant albumin.

In some aspects of the preceding method, the medium can further comprise dipeptidylpeptidase-4 (DPP-IV). In some aspects of the preceding method, the medium can further comprise recombinant DPP-IV.

In some aspects of the preceding method, the medium can further comprise DPP-IV secreted by the plurality of fibroblasts. In some aspects, the plurality of fibroblasts can be stimulated to express and/or secrete increased amounts of DPP-IV as compared to a plurality of fibroblasts cultured under the same conditions but left unstimulated. Accordingly, in some aspects, the preceding method can further comprise stimulating the plurality of fibroblasts to express and/or secrete increased amounts of DPP-IV.

In some aspects of the preceding method, the plurality of fibroblasts can be cultured in a medium comprising DA-DKP for at least about 1 week, or at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, including ranges between any two of the listed values, for example about 2 weeks to about 11 weeks, about 2 weeks to about 10 weeks, about 2 weeks to about 8 weeks, about 2 weeks to about 6 weeks, about 2 weeks to about 4 weeks, about 3 weeks to about 12 weeks, about 3 weeks to about 10 weeks, about 3 weeks to about 8 weeks about 3 weeks to about 6 weeks, about 4 weeks to about 12 weeks, about 4 weeks to about 10 weeks, about 4 weeks to about 8 weeks, about 4 weeks to about 6 weeks, about 6 weeks to about 12 weeks, about 6 weeks to about 10 weeks, or about 6 weeks to about 8 weeks.

The present disclosure provides a method of manufacturing ECM, the method comprising: a) culturing a plurality of fibroblasts in a medium comprising serum; b) gradually reducing the concentration of serum in the medium over a time period such that there is a reduction in the concentration of serum; c) culturing the plurality of fibroblasts in a medium comprising DA-DKP for a time period sufficient for the plurality of fibroblasts to produce extracellular matrix (ECM).

In some aspects of the preceding method, the medium comprising serum in step (a) can further comprise dipeptidylpeptidase-4 (DPP-IV). In some aspects, the medium comprising serum in step (a) can further comprise recombinant DPP-IV.

In some aspects of the preceding method, the medium comprising serum in step (a) can further comprise DPP-IV secreted by the plurality of fibroblasts. In some aspects, the plurality of fibroblasts can be stimulated to express and/or secrete increased amounts of DPP-IV as compared to a plurality of fibroblasts cultured under the same conditions but left unstimulated. Accordingly, in some aspects of the preceding method, step (a) can further comprise stimulating the fibroblasts to express and/or secrete increased amounts of DPP-IV. In some aspects, the preceding method can further comprise stimulating the plurality of fibroblasts to express and/or secrete increased amounts of DPP-IV. In some aspects, the stimulation can be performed prior to step (a). In some aspects, the stimulation can be performed prior to step (c).

In some aspects of the preceding method, steps (b) and (c) can be performed sequentially, in any order.

In some aspects of the preceding method, steps (b) and (c) can be performed concurrently.

In some aspects of the preceding method, the medium comprising DA-DKP in step (c) can further comprise albumin. In some aspects of the preceding method, the medium comprising DA-DKP in step (c) can further comprise recombinant albumin. As would be appreciated by the skilled artisan, recombinant serum albumin is albumin protein that is produced using a recombinant expression system, including, but not limited to, mammalian recombinant expression systems, insect recombinant expression systems, yeast recombinant expression systems, bacterial recombinant expression systems, algal recombinant expression systems and cell-free recombinant expression systems.

In some aspects of the preceding method, the medium comprising DA-DKP in step (c) can further comprise dipeptidylpeptidase-4 (DPP-IV). In some aspects, the medium comprising DA-DKP in step (c) can further comprise recombinant DPP-IV.

In some aspects of the preceding method, the medium comprising DA-DKP in step (c) can further comprise DPP-IV secreted by the plurality of fibroblasts. In some aspects, the plurality of fibroblasts can be stimulated to express and/or secrete increased amounts of DPP-IV as compared to a plurality of fibroblasts cultured under the same conditions but left unstimulated. Accordingly, in some aspects, the preceding method can further comprise stimulating the plurality of fibroblasts to express and/or secrete increased amounts of DPP-IV.

In some aspects of the preceding method, the plurality of fibroblasts can be cultured in a medium comprising serum for at least about 1 week, or at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, including ranges between any two of the listed values, for example about 2 weeks to about 11 weeks, about 2 weeks to about 10 weeks, about 2 weeks to about 8 weeks, about 2 weeks to about 6 weeks, about 2 weeks to about 4 weeks, about 3 weeks to about 12 weeks, about 3 weeks to about 10 weeks, about 3 weeks to about 8 weeks about 3 weeks to about 6 weeks, about 4 weeks to about 12 weeks, about 4 weeks to about 10 weeks, about 4 weeks to about 8 weeks, about 4 weeks to about 6 weeks, about 6 weeks to about 12 weeks, about 6 weeks to about 10 weeks, or about 6 weeks to about 8 weeks. In some aspects, the plurality of fibroblasts can be cultured in a medium comprising serum for a time period sufficient to produce mature ECM.

In some aspects of the preceding method, the plurality of fibroblasts can be cultured in a medium comprising DA-DKP for at least about 1 week, or at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, including ranges between any two of the listed values, for example about 2 weeks to about 11 weeks, about 2 weeks to about 10 weeks, about 2 weeks to about 8 weeks, about 2 weeks to about 6 weeks, about 2 weeks to about 4 weeks, about 3 weeks to about 12 weeks, about 3 weeks to about 10 weeks, about 3 weeks to about 8 weeks about 3 weeks to about 6 weeks, about 4 weeks to about 12 weeks, about 4 weeks to about 10 weeks, about 4 weeks to about 8 weeks, about 4 weeks to about 6 weeks, about 6 weeks to about 12 weeks, about 6 weeks to about 10 weeks, or about 6 weeks to about 8 weeks.

Performing a gradual reduction in serum may also be referred to herein as “serum weaning.”

In some aspects, gradually reducing the concentration of serum in the medium over a time period such that there is a reduction in the concentration of serum can comprise reducing the amount of serum in the medium over the time period until the concentration of serum in the medium is not more than about 5% (v/v). In some aspects, gradually reducing the concentration of serum in the medium over a time period such that there is a reduction in the concentration of serum can comprise reducing the amount of serum in the medium over the time period until the concentration of serum in the medium is not more than about 5% (v/v), or not more than about 4.5% (v/v), or not more than about 4% (v/v), or not more than about 3.5% (v/v), or not more than about 3% (v/v), or not more than about 2.5% (v/v), or not more than about 2% (v/v), or not more than about 1.5% (v/v), or not more than about 1% (v/v), or not more than about 0.5% (v/v), or not more than about 0.25% (v/v).

In some aspects, gradually reducing the amount of serum in the medium can take place over a time period of days, for example at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, including ranges between any two of the listed values, for example about 1-30 days, 1-20 days, 1-14 days, 1-10 days, 1-7 days, 1-5 days, 1-30 days, 2-20 days, 2-14 days, 2-10 days, 2-7 days, 2-5 days, 3-20 days, 3-14 days, 3-10 days, 3-7 days, 3-5 days, 5-20 days, 5-14 days, 5-10 days, 5-7 days, 7-20 days, 7-14 days, 7-10 days, 10-20 days, or 10-14 days.

In some aspects, gradually reducing the concentration of serum in the medium over a time period such that there is a reduction in the concentration of serum can comprise reducing the amount of serum in the medium over the time period until the concentration of serum in the medium is not more than about 5% of the original concentration of serum. In some aspects, gradually reducing the concentration of serum in the medium over a time period such that there is a reduction in the concentration of serum can comprise reducing the amount of serum in the medium over the time period until the concentration of serum in the medium is not more than about 5%, or not more than about 4.5%, or not more than about 4%, or not more than about 3.5%, or not more than about 3%, or not more than about 2.5%, or not more than about 2%, or not more than about 1.5%, or not more than about 1%, or not more than about 0.5%, or not more than about 0.25% of the original concentration of serum.

In some aspects, gradually reducing the concentration of serum in the medium over a time period such that there is a reduction in the concentration of serum can comprise reducing the amount of serum in the medium over the time period such that there is at least a 95% reduction in the concentration of serum. In some aspects, gradually reducing the concentration of serum in the medium over a time period such that there is a reduction in the concentration of serum can comprise reducing the amount of serum in the medium over the time period such that there is at least a 95% reduction, or at least a 97.5%, or at least a 99%, or at least a 99.5%, or at least a 99.95% reduction in the concentration of serum.

In some aspects, gradually reducing the amount of serum can be performed without cell expansion or cell subculture. In some aspects, gradually reducing the amount of serum can be performed without cell subculture.

In some aspects, gradually reducing the concentration of serum in the medium can be performed using any method known in the art. In a non-limiting example, gradually reducing the concentration of serum in the medium over a time period such that there is a reduction in the concentration of serum can comprise adding one or more amounts of serum-free medium such that the serum is diluted. In another non-limiting example, the gradual reduction of serum can comprise removal of one or more amounts of serum-containing medium and replacing with one or more amounts of serum-fee medium. Thus, without wishing to be bound by theory, by performing multiple rounds of replacing a portion of serum-containing medium with serum-free medium, the serum concentration can be gradually reduced. In some aspects, the one or more amounts of serum-free medium that is being used to replace the serum-containing medium can comprise DA-DKP.

In some aspects of the preceding methods, stimulating the plurality of fibroblasts to express and/or secrete increased amounts of DPP-IV can comprise contacting the fibroblasts with DA-DKP.

In some aspects of the preceding methods, stimulating the plurality of fibroblasts to express and/or secrete increased amounts of DPP-IV can comprise contacting the fibroblasts with one or more cytokines, wherein the one or more cytokines are selected from any cytokine appreciated in the art to increase the expression and/or secretion of DPP-IV in fibroblasts. In some aspects, the one or more cytokines can be selected from VEGF, Follistatin IL-6, IL-8, TGF-β, and MIP-1α.

In some aspects of the preceding methods, stimulating the plurality of fibroblasts to express and/or secrete increased amounts of DPP-IV can comprise genetically modifying the fibroblasts. Genetic modification can comprise any genetic modification method appreciated in the art that results in the increased expression and/or secretion of DPP-IV. Non-limiting examples of genetic modification include, but are not limited to, transfecting and/or transducing the fibroblasts with an expression vector comprising DPP-IV. In some aspects, the expression vector can be, but is not limited to, a viral vector or a plasmid.

In some aspects of the preceding methods, stimulating the plurality of fibroblasts to express and/or secrete increased amounts of DPP-IV can comprise contacting the fibroblasts with a conditioned medium. In some aspects, the conditioned medium can be medium that was previously used to culture mammalian cells.

In some aspects, the expression and/or secretion of an increased amount of DPP-IV describes the situation in which a fibroblast which is stimulated expresses and/or secretes DPP-IV in an amount that is greater than a fibroblast subjected to the same conditions but that is left unstimulated.

In some aspects, the preceding methods can further comprise isolating the ECM from the fibroblasts. In some aspects, isolating the ECM comprises separating the ECM from a substrate or solid phase

Any of the preceding methods can further comprise purifying the ECM. In some aspects, purifying the ECM comprises washing the ECM produced by the fibroblasts with an acidic buffer, contacting the ECM with dextranase or any combination thereof.

In some aspects of the preceding methods, the amount of ECM that is produced is at least about 10%, or at least about 20%, or at least about 30%, or at least about 40%, or at least about 50%, or at least about 60%, or at least about 70%, or at least about 80%, or at least about 90%, or at least about 100%, or at least about 200%, or at least about 300%, or at least about 400%, or at least about 500%, or at least about 1000% greater than the amount of ECM that is produced under control conditions.

In some aspects, control conditions comprise otherwise identical culturing conditions except for the omission of DA-DKP.

In some aspects of the preceding methods, the amount of ECM that is produced is at least 2 times, or at least about 3 times, or at least about 4 times, or at least about 5 times, or at least about 6 times, or at least about 7 times, or at least about 8 times, or at least about 9 times, or at least about 10 times greater than the amount of ECM that is produced under otherwise identical conditions except for the omission of DA-DKP.

Compositions

The present disclosure provides compositions comprising ECM produced using any of the methods described herein. In some aspects, the composition can further comprise DA-DKP. In some aspects, the composition can further comprise conditioned media used to culture the fibroblasts during the production of the ECM. In some aspects, the composition can further comprise fibroblasts.

The present disclosure provides pharmaceutical compositions comprising ECM produced using any of the methods described herein. In some aspects, a pharmaceutical composition can comprise at least one pharmaceutically acceptable carrier and/or excipient. In some aspects, the pharmaceutical composition can further comprise DA-DKP. In some aspects, the pharmaceutical composition can further comprise conditioned media used to culture the fibroblasts during the production of the ECM. In some aspects, the pharmaceutical composition can further comprise fibroblasts.

The present disclosure provides a therapeutic composition comprising ECM produced using any of the methods described herein. In some aspects, the therapeutic composition can further comprise DA-DKP. In some aspects, the therapeutic composition can further comprise conditioned media used to culture the fibroblasts during the production of the ECM. In some aspects, the therapeutic composition can further comprise fibroblasts.

The present disclosure provides a cosmetic composition comprising ECM produced using any of the methods described herein. In some aspects, the cosmetic composition can further comprise DA-DKP. In some aspects, the cosmetic composition can further comprise conditioned media used to culture the fibroblasts during the production of the ECM. In some aspects, the cosmetic composition can further comprise fibroblasts.

The present disclosure provides a composition comprising a plurality of fibroblasts, wherein the fibroblasts have been contacted with a cell culture medium comprising DA-DKP.

The present disclosure provides a composition comprising a conditioned cell medium, wherein the conditioned cell medium is the product of one or more of the culturing steps recited in the methods of the present disclosure.

The present disclosure provides a cell culture medium comprising DA-DKP. In some aspects, a cell culture medium can further comprise at least one growth factor and/or cytokines. In some aspects, the at least one growth factor and/or cytokine is selected from VEGF, Follistatin IL-6, IL-8, TGF-β, and MIP-1α.

In some aspects of the methods, compositions and kits of the present disclosure, the at least one growth factor and/or cytokine can be present in a medium at a concentration of at least about 1 nM, or at least about 5 nM, or at least about 10 nM, at least about 15 nM, or at least about 20 nM, or at least about 25 nM, or at least about 30 nM, or at least about 35 nM, or at least about 40 nM, or at least about 45 nM, or at least about 50 nM, or at least about 55 nM, or at least about 60 nM, or at least about 65 nM, or at least about 70 nM, or at least about 75 nM, or at least about 80 nM, or at least about 85 nM, or at least about 90 nM, or at least about 95 nM, or at least about 100 nM, or at least about 125 nM, or at least about 150 nM, or at least about 175 nM, or at least about 200 nM, or at least about 225 nM, or at least about 250 nM, or at least about 275 nM, or at least about 300 nM, or at least about 325 nM, or at least about 350 nM, or at least about 375 nM, or at least about 400 nM, or at least about 425 nM, or at least about 450 nM, or at least about 475 nM, or at least about 500 nM, or at least about 525 nM, or at least about 550 nM, or at least about 575 nM, or at least about 600 nM, or at least about 625 nM, or at least about 650 nM, or at least about 675 nM, or at least about 700 nM, or at least about 725 nM, or at least about 750 nM, or at least about 775 nM, or at least about 800 nM, or at least about 825 nM, or at least about 850 nM, or at least about 875 nM, or at least about 900 nM, or at least about 925 nM, or at least about 950 nM, or at least about 975 nM, or at least about 1000 nM, including ranges between any two of the listed values.

In some aspects of the methods, compositions and kits of the present disclosure, the at least one growth factor and/or cytokine can be present in a medium at a concentration of about 1 nM, or about 5 nM, or about 10 nM, about 15 nM, or about 20 nM, or about 25 nM, or about 30 nM, or about 35 nM, or about 40 nM, or about 45 nM, or about 50 nM, or about 55 nM, or about 60 nM, or about 65 nM, or about 70 nM, or about 75 nM, or about 80 nM, or about 85 nM, or about 90 nM, or about 95 nM, or about 100 nM, or about 125 nM, or about 150 nM, or about 175 nM, or about 200 nM, or about 225 nM, or about 250 nM, or about 275 nM, or about 300 nM, or about 325 nM, or about 350 nM, or about 375 nM, or about 400 nM, or about 425 nM, or about 450 nM, or about 475 nM, or about 500 nM, or about 525 nM, or about 550 nM, or about 575 nM, or about 600 nM, or about 625 nM, or about 650 nM, or about 675 nM, or about 700 nM, or about 725 nM, or about 750 nM, or about 775 nM, or about 800 nM, or about 825 nM, or about 850 nM, or about 875 nM, or about 900 nM, or about 925 nM, or about 950 nM, or about 975 nM, or about 1000 nM, including ranges between any two of the listed values.

In some aspects of the methods, compositions and kits of the present disclosure, the at least one growth factor and/or cytokine can be present in a medium at a concentration of at least about or at least about 5 μM, or at least about 10 μM, at least about 15 μM, or at least about 20 μM, or at least about 25 μM, or at least about 30 μM, or at least about 35 μM, or at least about 40 μM, or at least about 45 μM, or at least about 50 μM, or at least about 55 μM, or at least about 60 μM, or at least about 65 μM, or at least about 70 μM, or at least about 75 μM, or at least about 80 μM, or at least about 85 μM, or at least about 90 μM, or at least about 95 μM, or at least about 100 μM, or at least about 125 μM, or at least about 150 μM, or at least about 175 μM, or at least about 200 μM, or at least about 225 μM, or at least about 250 μM, or at least about 275 μM, or at least about 300 μM, or at least about 325 μM, or at least about 350 μM, or at least about 375 μM, or at least about 400 μM, or at least about 425 μM, or at least about 450 μM, or at least about 475 μM, or at least about 500 μM, or at least about 525 μM, or at least about 550 μM, or at least about 575 μM, or at least about 600 μM, or at least about 625 μM, or at least about 650 μM, or at least about 675 μM, or at least about 700 μM, or at least about 725 μM, or at least about 750 μM, or at least about 775 μM, or at least about 800 μM, or at least about 825 μM, or at least about 850 μM, or at least about 875 μM, or at least about 900 μM, or at least about 925 μM, or at least about 950 μM, or at least about 975 μM, or at least about 1000 μM, including ranges between any two of the listed values.

In some aspects of the methods, compositions and kits of the present disclosure, the at least one growth factor and/or cytokine can be present in a medium at a concentration of about 1 μM, or about 5 μM, or about 10 μM, about 15 μM, or about 20 μM, or about 25 μM, or about 30 μM, or about 35 μM, or about 40 μM, or about 45 μM, or about 50 μM, or about 55 μM, or about 60 μM, or about 65 μM, or about 70 μM, or about 75 μM, or about 80 μM, or about 85 μM, or about 90 μM, or about 95 μM, or about 100 μM, or about 125 μM, or about 150 μM, or about 175 μM, or about 200 μM, or about 225 μM, or about 250 μM, or about 275 μM, or about 300 μM, or about 325 μM, or about 350 μM, or about 375 μM, or about 400 μM, or about 425 μM, or about 450 μM, or about 475 μM, or about 500 μM, or about 525 μM, or about 550 μM, or about 575 μM, or about 600 μM, or about 625 μM, or about 650 μM, or about 675 μM, or about 700 μM, or about 725 μM, or about 750 μM, or about 775 μM, or about 800 μM, or about 825 μM, or about 850 μM, or about 875 μM, or about 900 μM, or about 925 μM, or about 950 μM, or about 975 μM, or about 1000 μM, including ranges between any two of the listed values.

In some aspects of the methods, compositions and kits of the present disclosure, the at least one growth factor and/or cytokine can be present in a medium at a concentration of at least about 1 mM, or at least about 2 mM, or at least about 3 mM, or at least about 4 mM, or at least about 5 mM, or at least about 6 mM, or at least about 7 mM, or at least about 8 mM, or at least about 9 mM, or at least about 10 mM including ranges between any two of the listed values.

Kits

The present disclosure provides kits comprising one or more of the compositions of the present disclosure.

The present disclosure provides kits comprising a plurality of fibroblasts and at least one cell culture medium comprising DA-DKP.

The present disclosure provides kits comprising at least one pharmaceutical composition of the present disclosure.

The present disclosure provides kits comprising at least one cosmetic composition of the present disclosure.

The present disclosure provides kits comprising at least one therapeutic composition of the present disclosure.

In some aspects, a kit can comprise a container that holds ECM produced using the methods of the present disclosure.

In some aspects, a kit can comprise instructions for use. In some aspect, the instructions can be written instructions.

Uses of the Compositions of the Present Disclosure

The compositions of the present disclosure can be used for cosmetic purposes. Cosmetic purposes include, but are not limited to, facial wrinkle filling, reduction of visible aging signs, promotion of hair growth, improved appearance of skin or any combination thereof.

Accordingly, the present disclosure provides a method of filling facial wrinkles, reducing visible aging signs, promoting hair growth, improving appearance of skin or any combination thereof, the method comprising administering to a subject at least one effective amount of at least one composition of the present disclosure. The present disclosure provides at least one composition of the present disclosure for use in filling facial wrinkles, reducing visible aging signs, promoting hair growth, improving appearance of skin or any combination thereof. The present disclosure provides the use of at least one composition of the present disclosure in the manufacture of a medicament for the filling of facial wrinkles, reduction of visible aging signs, promotion of hair growth, improvement in the appearance of skin or any combination thereof.

The compositions of the present disclosure can be used for therapeutic purposes. Accordingly, the present disclosure provides methods of treating and/or preventing at least one disease or disorder, the method comprising administering at least one therapeutically effective amount of at least one composition of the present disclosure. The present disclosure provides at least one composition of the present disclosure for use in the treatment and/or prevention of at least one disease and/or disorder in a subject. The present disclosure provides the use of at least one composition of the present disclosure in the manufacture of a medicament for treating and/or preventing at least one disease and/or disorder in a subject.

In some aspects, the disease and/or disorder can be at least one of a cancer, a musculoskeletal disorder, an orthopedic dysfunction, pain, cardiovascular disorder, cutaneous disease, surgical wounds, solid-tumors requiring treatment with surgical excision, osteochondral defects, osteoarthritis, degenerative disc-disease, surgical wounds required for orthopedics, surgical wounds associated with tumor resection cavities, arthritis, autoimmune disorders and rheumatoid arthritis.

Additional uses for the compositions of the present disclosure include, but are not limited to, treating tissues in patients suffering from musculoskeletal disorders, treating orthopedic dysfunction and associated pain, treating cardiovascular disorders, treating cutaneous diseases, treating age-related cosmetic skin and hair conditions requiring improvement in appearance, treating surgical wounds, treating solid-tumors requiring treatment including surgical excision, treating chemotherapy-induced adverse effects, use as an immunotherapy, improving medical devices for musculoskeletal applications, as a biologic in musculoskeletal applications including osteochondral defect repair, treating osteoarthritis, treating degenerative disc-disease, treating surgical wounds required for orthopedics, treating surgical wounds associated with tumor resection cavities, use in cardiovascular regeneration devices and biologics, use in cutaneous wound devices and biologics, use as dermal fillers for treating wrinkles, use as a topical cosmetic, use in therapeutic hair growth, and use as a cell-delivery and/or drug-delivery vehicle for increased persistence and reduced unwanted immune reactions when transferred to patient.

Aspartyl-alanyl-diketopiperazine (DA-DKP)

As would be appreciated by the skilled artisan, aspartyl-alanyl-diketopiperazine (DA-DKP) has the following chemical structure:

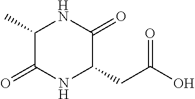

As used herein, the terms “aspartyl-alanyl-diketopiperazine” and “DA-DKP” can refer to any salt form of DA-DKP, including, but not limited to, pharmaceutically acceptable salts.

The DA-DKP used in the methods, kits and compositions of the present disclosure can be produced using any method known in appreciated in the art.

In a non-limiting example, DA-DKP can be produced by isolating DA-DKP from a solution comprising serum albumin. In some aspects, the solution comprising serum albumin further comprises at least one endopeptidase. In some aspects, the solution comprising serum albumin further comprises DPP-IV. The DA-DKP can be isolated from the solution using methods standard in the art.

In some aspects, DA-DKP can be produced by isolating DA-DKP from a conditioned medium that was used to culture a plurality of mammalian cells.

In some aspects, DA-DKP can be produced by isolating DA-DKP from serum. In some aspects, the serum can be a mammalian serum. In some aspects, the serum can be bovine serum or fetal bovine serum. In some aspects, the serum can be human serum. In some aspects, the serum can be one or more of human serum, bovine serum, goat serum, rat serum, goat serum, porcine serum, chicken serum, chicken egg serum, mouse serum, rabbit serum, sheep serum or any other serum known in the art.

In some aspects, DA-DKP can be chemically synthesized using methods standard in the art.

In some aspects of the methods, compositions and kits of the present disclosure, DA-DKP can be present in a medium at a concentration of at least about 1 nM, or at least about 5 nM, or at least about 10 nM, at least about 15 nM, or at least about 20 nM, or at least about 25 nM, or at least about 30 nM, or at least about 35 nM, or at least about 40 nM, or at least about 45 nM, or at least about 50 nM, or at least about 55 nM, or at least about 60 nM, or at least about 65 nM, or at least about 70 nM, or at least about 75 nM, or at least about 80 nM, or at least about 85 nM, or at least about 90 nM, or at least about 95 nM, or at least about 100 nM, or at least about 125 nM, or at least about 150 nM, or at least about 175 nM, or at least about 200 nM, or at least about 225 nM, or at least about 250 nM, or at least about 275 nM, or at least about 300 nM, or at least about 325 nM, or at least about 350 nM, or at least about 375 nM, or at least about 400 nM, or at least about 425 nM, or at least about 450 nM, or at least about 475 nM, or at least about 500 nM, or at least about 525 nM, or at least about 550 nM, or at least about 575 nM, or at least about 600 nM, or at least about 625 nM, or at least about 650 nM, or at least about 675 nM, or at least about 700 nM, or at least about 725 nM, or at least about 750 nM, or at least about 775 nM, or at least about 800 nM, or at least about 825 nM, or at least about 850 nM, or at least about 875 nM, or at least about 900 nM, or at least about 925 nM, or at least about 950 nM, or at least about 975 nM, or at least about 1000 nM, including ranges between any two of the listed values.

In some aspects of the methods, compositions and kits of the present disclosure, DA-DKP can be present in a medium at a concentration of about 1 nM, or about 5 nM, or about 10 nM, about 15 nM, or about 20 nM, or about 25 nM, or about 30 nM, or about 35 nM, or about 40 nM, or about 45 nM, or about 50 nM, or about 55 nM, or about 60 nM, or about 65 nM, or about 70 nM, or about 75 nM, or about 80 nM, or about 85 nM, or about 90 nM, or about 95 nM, or about 100 nM, or about 125 nM, or about 150 nM, or about 175 nM, or about 200 nM, or about 225 nM, or about 250 nM, or about 275 nM, or about 300 nM, or about 325 nM, or about 350 nM, or about 375 nM, or about 400 nM, or about 425 nM, or about 450 nM, or about 475 nM, or about 500 nM, or about 525 nM, or about 550 nM, or about 575 nM, or about 600 nM, or about 625 nM, or about 650 nM, or about 675 nM, or about 700 nM, or about 725 nM, or about 750 nM, or about 775 nM, or about 800 nM, or about 825 nM, or about 850 nM, or about 875 nM, or about 900 nM, or about 925 nM, or about 950 nM, or about 975 nM, or about 1000 nM, including ranges between any two of the listed values.

In some aspects of the methods, compositions and kits of the present disclosure, DA-DKP can be present in a medium at a concentration of at least about 1 μM, or at least about 5 μM, or at least about 10 μM, at least about 15 μM, or at least about 20 μM, or at least about 25 μM, or at least about 30 μM, or at least about 35 μM, or at least about 40 μM, or at least about 45 μM, or at least about 50 μM, or at least about 55 μM, or at least about 60 μM, or at least about 65 μM, or at least about 70 μM, or at least about 75 μM, or at least about 80 μM, or at least about 85 μM, or at least about 90 μM, or at least about 95 μM, or at least about 100 μM, or at least about 125 μM, or at least about 150 μM, or at least about 175 μM, or at least about 200 μM, or at least about 225 μM, or at least about 250 μM, or at least about 275 μM, or at least about 300 μM, or at least about 325 μM, or at least about 350 μM, or at least about 375 μM, or at least about 400 μM, or at least about 425 μM, or at least about 450 μM, or at least about 475 μM, or at least about 500 μM, or at least about 525 μM, or at least about 550 μM, or at least about 575 μM, or at least about 600 μM, or at least about 625 μM, or at least about 650 μM, or at least about 675 μM, or at least about 700 μM, or at least about 725 μM, or at least about 750 μM, or at least about 775 μM, or at least about 800 μM, or at least about 825 μM, or at least about 850 μM, or at least about 875 μM, or at least about 900 μM, or at least about 925 μM, or at least about 950 μM, or at least about 975 μM, or at least about 1000 μM, including ranges between any two of the listed values.

In some aspects of the methods, compositions and kits of the present disclosure, DA-DKP can be present in a medium at a concentration of about 1 μM, or about 5 μM, or about 10 μM, about 15 μM, or about 20 μM, or about 25 μM, or about 30 μM, or about 35 μM, or about 40 μM, or about 45 μM, or about 50 μM, or about 55 μM, or about 60 μM, or about 65 μM, or about 70 μM, or about 75 μM, or about 80 μM, or about 85 μM, or about 90 μM, or about 95 μM, or about 100 μM, or about 125 μM, or about 150 μM, or about 175 μM, or about 200 μM, or about 225 μM, or about 250 μM, or about 275 μM, or about 300 μM, or about 325 μM, or about 350 μM, or about 375 μM, or about 400 μM, or about 425 μM, or about 450 μM, or about 475 μM, or about 500 μM, or about 525 μM, or about 550 μM, or about 575 μM, or about 600 μM, or about 625 μM, or about 650 μM, or about 675 μM, or about 700 μM, or about 725 μM, or about 750 μM, or about 775 μM, or about 800 μM, or about 825 μM, or about 850 μM, or about 875 μM, or about 900 μM, or about 925 μM, or about 950 μM, or about 975 μM, or about 1000 μM, including ranges between any two of the listed values.

In some aspects of the methods, compositions and kits of the present disclosure, DA-DKP can be present in a medium at a concentration of at least about 1 mM, or at least about 2 mM, or at least about 3 mM, or at least about 4 mM, or at least about 5 mM, or at least about 6 mM, or at least about 7 mM, or at least about 8 mM, or at least about 9 mM, or at least about 10 mM including ranges between any two of the listed values.

In some aspects of the methods, compositions and kits of the present disclosure, DA-DKP can be present in a medium at a concentration of about 1 mM, or about 2 mM, or about 3 mM, or about 4 mM, or about 5 mM, or about 6 mM, or about 7 mM, or about 8 mM, or about 9 mM, or about 10 mM including ranges between any two of the listed values.

In some aspects of the methods, compositions and kits of the present disclosure DA-DKP can be present in a medium at a concentration of about 1 nM to about 1 μM, about 1 μM to about 1 mM, or about 1 μM to about 10 mM, or about 1 mM to about 10 mM, or about 10 μM to about 10 mM, or about 100 μM to about 10 mM.

In aspects wherein a medium comprises DA-DKP, the medium can further comprise N-actyl DL-tryptophan. In some aspects, the N-actyl DL-tryptophan can be present in concentrations greater than about 1 mM.

In aspects wherein a medium comprises DA-DKP, the medium can further comprise caprylic acid and/or caprylate stabilizers. In some aspects, the caprylic acid and/or caprylate stabilizers can be present in concentrations greater than about 1 mM.

Dipeptidylpeptidase-4 (DPP-IV)

As would be appreciated by the skilled artisan, Dipeptidylpeptidase-4 is a protein that is commonly referred to as one of Dipeptidylpeptidase-4, DPP-IV, DPP4, ADABP, ADCP2, CD26, DPPIV and TP103. Accordingly, all of these terms are used interchangeably herein.

In some aspects of the methods, compositions and kits of the present disclosure, DPP-IV can be human DPP-IV. In some aspects, DPP-IV can be an orthologue, paralogue or homologue of the human DDP-IV protein derived from a species other than human.

Serum Albumin

In some aspects of the methods, kits and compositions of the present disclosure, serum albumin can be serum albumin which is isolated from an animal source.

In some aspects, the serum albumin can be mammalian serum albumin.

In some aspects, the serum albumin can be human serum albumin.

In some aspects, the serum albumin can be bovine serum albumin.

In some aspects, the serum albumin can be one or more of human serum albumin, bovine serum albumin, goat serum albumin, rat serum albumin, goat serum albumin, porcine serum albumin, chicken serum albumin, chicken egg serum albumin, mouse serum albumin, rabbit serum albumin, sheep serum albumin or any other serum albumin known in the art.

In some aspects of the methods, kits and compositions of the present disclosure, serum albumin can be recombinant serum albumin which is produced using a recombinant expression system. As would be appreciated by the skilled artisan, examples of recombinant expression systems include, but are not limited to, mammalian recombinant expression systems, insect recombinant expression systems, yeast recombinant expression systems, bacterial recombinant expression systems, algal recombinant expression systems and cell-free recombinant expression systems.

In some aspects, the recombinant serum albumin can be produced from a rice recombinant expression system. In some aspects, the recombinant serum albumin can be produced from a yeast recombinant expression system.

Recombinant serum albumin can be recombinant human serum albumin, recombinant bovine serum albumin, recombinant goat serum albumin, recombinant rat serum albumin, recombinant goat serum albumin, recombinant porcine serum albumin, recombinant chicken serum albumin, recombinant chicken egg serum albumin, recombinant mouse serum albumin, recombinant rabbit serum albumin, recombinant sheep serum albumin, recombinant rice albumin

In some aspects of the methods, compositions and kits of the present disclosure, serum albumin can be present in a medium at a concentration of at least about 0.1 g/L, or at least about 0.5 g/L, or at least about 1 g/L, or at least about 1.5 g/L, or at least about 2 g/L, or at least about 2.5 g/L, or at least about 3 g/L, or at least about 3.5 g/L, or at least about 4 g/L, or at least about 4.5 g/L, or at least about 5 g/L, or at least about 5.5 g/L, or at least about 6.5 g/L, or at least about 7 g/L, or at least about 7.5 g/L, or at least about 8 g/L, or at least about 8.5 g/L, or at least about 9 g/L, or at least about 9.5 g/L, or at least about 10 g/L, or at least about 15 g/L, or at least about 20 g/L, including ranges between any two of the listed values.

In some aspects of the methods, compositions and kits of the present disclosure, serum albumin can be present in a medium at a concentration of about 0.1 g/L, or about 0.5 g/L, or about 1 g/L, or about 1.5 g/L, or about 2 g/L, or about 2.5 g/L, or about 3 g/L, or about 3.5 g/L, or about 4 g/L, or about 4.5 g/L, or about 5 g/L, or about 5.5 g/L, or about 6.5 g/L, or about 7 g/L, or about 7.5 g/L, or about 8 g/L, or about 8.5 g/L, or about 9 g/L, or about 9.5 g/L, or about 10 g/L, or about 15 g/L, or about 20 g/L, including ranges between any two of the listed values.

Fibroblasts

“Fibroblast” used herein in accordance with its ordinary meaning in the field, and includes a class of cells that provide structural framework (stroma) for a variety of animal tissues. Fibroblasts can also migrate at the site of a wound to mediate wound healing, and can be a component in a variety of connective tissues. Fibroblasts can be identified, for example, using fibroblast-specific antibodies, for example antibody TE-7, described in Goodpaster et al. (2008), J. Histochem Cyotchem. 56: 347-58, which is hereby incorporated by reference in its entirety.

In some aspects, the plurality of fibroblasts can comprise a plurality of activated fibroblasts.

As would be appreciated by the skilled artisan, the term “activated fibroblasts” refers to fibroblast cells that are actively producing extracellular matrix (ECM). As would be appreciated by the skilled artisan, activated fibroblasts express elevated levels of Fibroblast Activation Protein (FAP).

In some aspects, activated fibroblasts secrete at least one cytokine selected from VEGF, Follistatin IL-6, IL-8, TGF-β, and MIP-1α.

In some aspects, activated fibroblasts express elevated levels of at least one of: a) Fibroblast Activation Protein (FAP); b) at least one cytokine, wherein the at least one cytokine is selected from IL-6, IL-8, TGF-β and MIP-1α; and c) at least one ECM protein, wherein the ECM protein is selected from a laminin, fibronectin, collagen type I, collagen type II, collagen type III, collagen type IV, collagen type V, collagen type VI. In some aspects, the elevated levels are levels that are higher than fibroblasts that are not actively producing ECM and/or levels that are higher than those found in fibrocytes.

As would be appreciated by the skilled artisan, the amount of biomarkers (e.g. Fibroblast Activation Protein) and/or cytokines (e.g. VEGF, Follistatin IL-6, IL-8, TGF-β, and MIP-1α.) can be quantified using methods that are standard in the art, including, but not limited to, Western Blotting, Mass Spectrometry, ELISA, PCR, quantitative PCR, reverse transcription quantitative PCR, immunohistochemistry techniques or any other method known in the art for the quantification of biomarkers and/or cytokines.

In some aspects, activated fibroblasts secrete at least one ECM protein selected from a laminin, fibronectin, or collagen type I, collagen type II, collagen type III, collagen type IV, collagen type V, collagen type VI.

In some aspects, fibroblasts used in the methods, compositions and kits of the present disclosure can be obtained by differentiating induced Pluripotent Stem cells (iPSCs) into fibroblasts. As would be appreciated by the skilled artisan, iPSCs can be differentiated into fibroblasts using methods known in the art. In some aspects, the IPSCs can be from a single donor.

In some aspects, fibroblasts used in the methods, compositions and kits of the present disclosure can be obtained by differentiating embryonic stem (ES) cells into fibroblasts. As would be appreciated by the skilled artisan, ES cells can be differentiated into fibroblasts using methods known in the art. In some aspects, the ES cells can be from a single donor.

Without wishing to be bound by theory, iPSCs and/ES cells from a single donor can offer safety advantages, for example limiting the exposure of the cells to only a single donor's complement of viruses, microbial organisms, or other potential pathogens, and thus minimizing the risk for transmission of disease compared to collections of cells from multiple donors.

In some aspects, fibroblasts can be obtained from adult dermal biopsies.

Extracellular Matrix

“Extracellular Matrix” (ECM) is used herein in accordance with its ordinary meaning in the field, and includes molecules secreted by cells such as proteins and carbohydrates, which provide a structure to support the cells. The ECM can comprise fibrillar proteins such as collagen. Human ECM can include a number of collagen proteins, including, for example COIL, COL3, COL4, COL5, and COL6, as well as combinations of these proteins.

In some aspects, the ECM that is produced using the methods of the present disclosure can comprise soluble ECM. In some aspects, the ECM that is produced using the methods of the present disclosure can comprise mature ECM. In some aspects, the ECM that is produced using the methods of the present disclosure can comprise soluble, mature ECM.

“Soluble” (for example in the context of soluble ECM), is used herein in accordance with its ordinary meaning in the field, and includes a type or fraction of ECM which is dissolved or can be dissolved in an aqueous phase. As such, soluble ECM can recovered in an aqueous phase and maintained in an aqueous phase. In some aspects, the soluble ECM does not precipitate in an aqueous phase. In some aspects, the soluble ECM can be maintained stably in an aqueous phase under the same conditions for at least 24 hours, with minimal precipitation, for example so that about or less than about 10%, 9%, 8%, 7%, 6%, 5%, 4%, 3%, or 1%, of the soluble ECM precipitates.

In some aspects, the ECM that is produced using the methods of the present disclosure can comprise soluble ECM and insoluble ECM. In some aspects, the ECM is at least about 80%, or at least about 85%, or at least about 90%, or at least about 95%, or at least about 99%, or at least about 99.5% soluble ECM.

In some aspects, the ECM that is produced using the methods of the present disclosure can comprise fibrillar collagenous ECM collagen, which is a mature ECM that comprises Collagen Type I (COL1) as major component, and can also further comprise Collagen Type III (COL3), Collagen Type IV (COL4), Collagen Type V (COL5), and/or Collagen Type VI (COL6), for example about 90% COL1 and about 10% COL3, COL4, COL5, and/or COL6, though other percentages of (i) COL1 and (ii) COL3, COL4, COL5 and/or COL6, are suitable, for example about 97% and 3% respectively, about 95% and 5% respectively, about 93% and 7% respectively, about 85% and 15% respectively, about 80% and 20% respectively, about 75% and 25% respectively, or about 70% and 30%, respectively.

In some aspects, soluble human ECM produced in accordance with the methods, compositions and kits of the present disclosure can comprise a greater amount of mature collagen type 1, for example a greater amount of mature triple-helical collagen type 1, as well as some less abundant fibrillar collagens, for example collagen types 3, 5, and 6.

“Mature ECM” is used herein in accordance with its ordinary meaning in the field, and includes cross-linked ECM, ECM that comprises a c-terminal propeptide of COL1, a triple-helical or non-reducible gamma-form fibrillary collagen, or a combination of two or more of these features. In some aspects, the mature ECM comprises a triple-helical and/or non-reducible gamma-form fibrillar collagen. In some aspects, the mature ECM comprises collagen. In some embodiments, about 90% (w/w) of the ECM comprises COL1, and about 10% is selected from the group consisting of: COL3, COL4, COL5, COL6, or a combination of any of these or all of these. In some aspects, at least about 80% of the mature ECM comprises COL1, and at least about 10% is selected from the group consisting of: COL3, COL4, COL5, COL6, and a combination of any of these. In some aspects, at least about 85% of the mature ECM comprises COL1 (for example, at least about 85%, 87%, 90%, or 95%), and at least about 5% (for example, at least about 5%, 10%, 13%, or 15%) is selected from the group consisting of: COL3, COL4, COL5, COL6, and a combination of any of these. In some embodiments, the mature ECM comprises a c-terminal propeptide of COL1, or a triple-helical or non-reducible gamma-form fibrillar collagen, or both. In some aspects, the mature ECM comprises a triple-helical and/or non-reducible gamma-form fibrillar collagen. It is noted that different molecules of mature ECM, for example collagen molecules as described herein, can be readily detected using an ELISA, among other assays. In some embodiments, an antibody specific for a collagen protein (or C-terminal propeptide of COL1) is used in a quantitative ELISA to ascertain amounts of components of mature ECM.

In some aspects, the ECM produced by the methods of the present disclosure can comprise large structures to the extent that some of the structures are visible under a microscope. In some aspects, the ECM that is produced using the methods of the present disclosure comprise nanostructures that have a greatest diameter of at least 200 nm, and up to 10,000 nm, for example at least 200 nm, 300 nm, 400 nm 500 nm, 1000 nm, 2000 nm, 5000 nm, or more. In some aspects, the nanostructures have a greatest diameter of 200 nm to 10,000 nm.

The ECM in accordance with methods, compositions, and kits of the present disclosure can comprise increased levels of commercially-useful mature collagens (such as triple-helical or non-reducible gamma-form fibrillar collagen, or both).

ECM, and in particular, human ECM products as produced according to methods, compositions, and kits of the present disclosure are useful for treating tissues in patients suffering from musculoskeletal disorders, orthopedic dysfunction and associated pain, cardiovascular disorders, cutaneous diseases, age-related cosmetic skin and hair conditions requiring improvement in appearance, surgical wounds, solid-tumors requiring treatment including surgical excision, chemotherapy and immunotherapy. Uses for the human ECM products produced by methods, kits, and compositions of the present disclosure, by way of example, include medical devices and biologics for musculoskeletal applications including osteochondral defect repair, osteoarthritis, degenerative disc-disease, surgical wounds for orthopedics; surgical wounds associated with tumor resection cavities, cardiovascular regeneration devices and biologics, cutaneous wound devices and biologics, dermal fillers for treating wrinkles, topical cosmetics, therapeutic hair growth, and cell-delivery and drug-delivery vehicles for increased persistence and reduction of unwanted immune reactions when transferred to a patient.

In some aspects, ECM (e.g., a human ECM product) produced according to the methods, compositions, and kits of the present disclosure can be used for at least one of a medical product, a cosmetic product, a drug, a medical device, a treatment, or a biologic.

In some aspects, the ECM produced according to the methods, compositions, and kits of the present disclosure can be substantially free xeno-free, in that the ECM is substantially free of contaminants which are foreign to the human body. In some aspects, the ECM produced according to the methods, compositions and kits of the present disclosure comprise no more than 3%, or no more than 2%, or no more than 1%, or no more than 0.5%, or no more than 0.25%, or no more than 0.1%, or no more than 0.01% contaminants which are foreign to the human body.

In some aspects, the methods of the present disclosure provide for the commercial scale production comprising production of at least 50 g of ECM, for example at least 50, 100, 200, 300, 400, 500, 600, 700, 800, 900, 1000, 1500, 2000, 3000, 4000, or 5000 g of ECM, including ranges between any two of the listed values.

Cell Culture and Substrates

As would be appreciated by the skilled artisan, a variety of approaches for cell culture can be used in methods, kits, and compositions of the present disclosure. Detailed guidance on protocols and reagents for cell culture can be found, for example, in Sambrook et al., “Molecular Cloning: A Laboratory Manual (Third ed.), Cold Spring Harbor Laboratory Press, Cold Spring Harbor, N.Y., 2000, which is hereby incorporated by reference in its entirety. Generally, culturing fibroblasts for the manufacture of ECMs according to methods, compositions, and kits of the present disclosure can be performed in culture medium.

In some aspects, a cell culture medium can comprise serum. In some aspects, the amount of serum in the culture medium (v/v) is about 0.1% to about 20%, for example about 0.1% to about 15%, about 0.1% to about 10%, about 0.1% to about 5%, about 0.1% to about 3%, about 0.1% to about 1%, about 1% to about 20%, about 1% to about 15%, about 1% to about 10%, about 1% to about 5%, about 1% to about 3%, about 3% to about 20%, about 3% to about 35%, about 3% to about 30%, about 3% to about 5%, about 5% to about 20%, about 5% to about 15%, about 5% to about 10%, about 10% to about 20%, or about 15% to about 20%, for example about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20%, including ranges between any two of the listed values. In some aspects, the amount of serum in the culture medium (v/v) is about 0.1% to about 10%.

In some aspects, fibroblasts can be cultured on or near a substrate. In some aspects, the substrate comprises, consists of, or consists essentially of dextran, for example dextran microcarriers. Without wishing to be bound by theory, dextran substrates can subsequently be digested using dextranase as described herein, which can facilitate isolation and purification of ECM produced by cell culture in accordance with the methods of the present disclosure. In some aspects, the substrate comprises, consists of or consists essentially of a carbohydrate. In some aspects, the substrate comprises, consists of, or consists essentially of a polymer. In some aspects, the substrate comprises, consists of, or consists essentially of a plastic. In some aspects, isolating the ECM comprises separating the ECM from any substrate or solid phase. In some aspects, the isolated ECM is separate from (e.g., not bound to) any substrate or solid phase. Without wishing to be bound by theory, it is contemplated that dextranase does not act as a protease on the ECM, and as such, isolating ECM using dextranase in accordance with some embodiments herein yields intact (non-protease-digested ECM). In some embodiments, the isolated ECM has not been digested by any protease, and is therefore intact.

As would be appreciated by the skilled artisan, dextran is a large, branched carbohydrate made out of many glucose molecules. Dextran chains can be of varying lengths, for example having molecular weights from as few as 3 kDa to more than 2000 kDa. As would be appreciated by the skilled artisan, Dextranase is a bacterial enzyme that catalyzes the following chemical reaction: endohydrolysis of (1->6)-alpha-D-glucosidic linkages in dextran.

In some aspects, any of the methods performed herein can be performed in a bioreactor.

In some aspects of the methods of the present disclosure, any of the culturing steps can be performed at a volume of at least about 50 liters, or at least about 100 liters, or at least about 150 liters, or at least about 200 liters, or at least about 250 liters, or at least about 300 liters, or at least about 350 liters, or at least about 400 liters, or at least about 450 liters, or at least about 500 liters, or at least about 550 liters, or at least about 600 liters, or at least about 650 liters, or at least about 700 liters, or at least about 750 liters, or at least about 800 liters, or at least about 850 liters, or at least about 900 liters, or at least about 950 liters, or at least about 1000 liters, or at least about 5000 liters, or at least about 10,000 liters, including ranges between any two of the listed values, for example 10-1000 liters, 10-500 liters, 10-200 liters, 50-1000 liters, 50-500 liters, 50-200 liters, 100-1000 liters, 100-500 liters, or 100-200 liters.

In some aspects of the methods of the present disclosure, cell culturing steps can be performed under conditions of normoxia.

Pharmaceutical Compositions

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

As used herein, the term “treating” or “treat” describes the management and care of a subject for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” “prevent,” or “protecting against” describes reducing or eliminating the onset of the symptoms or complications of such disease, condition or disorder.

The term “therapeutically effective amount”, as used herein, refers to an amount of a pharmaceutical agent, e.g., ECM, to treat, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. The dosage may vary within this range depending upon the dosage form employed and sensitivity of the subject.

As used herein, the term “therapeutic efficacy” of a composition refers to the ability of the composition to treat and/or prevent a disease and/or disorder described herein. Accordingly, in a non-limiting example, a composition that is said to have enhanced therapeutic efficacy is a composition that more effectively treats and/or prevents a disease and/or disorder, for example by being more potent (e.g. thereby allowing the administration of smaller amounts of the composition) and/or by eliciting fewer negative side effects (e.g. eliciting a reduced immune response)

As used herein, the term “cosmetic efficacy” of a composition refers to the ability of the composition to effectuate a desired cosmetic change. Non-limiting examples of cosmetic changes can include, but are not limited to, filling facial wrinkles, reducing visible aging signs, promoting hair growth, improving appearance of skin or any combination thereof. Accordingly, in a non-limiting example, a composition that is said to have enhanced cosmetic efficacy is composition that more effectively causes a desired cosmetic change, as described herein, for example by being (e.g. thereby allowing the administration of smaller amounts of the composition), by eliciting fewer negative side effects (e.g. eliciting a reduced immune response) and/or by causing a change that has not been observed through the use of a different composition.

Accordingly, in a non-limiting example, a composition that is said to have enhanced therapeutic efficacy is a composition that more effectively treats and/or prevents a disease and/or disorder, for example by being more potent (e.g. thereby allowing the administration of smaller amounts of the composition) and/or by eliciting fewer negative side effects (e.g. eliciting a reduced immune response).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme

In some aspects, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method of manufacturing Extracellular matrix (ECM), the method comprising culturing a plurality of fibroblasts in a medium comprising aspartyl-alanyl-diketopiperazine (DA-DKP) for a time period sufficient for the plurality of fibroblasts to produce extracellular matrix (ECM).

2. A method of manufacturing ECM, the method comprising:
a) culturing a plurality of fibroblasts in a medium comprising serum;
b) gradually reducing the concentration of serum in the medium over a time period such that there is a reduction in the concentration of serum; and
c) culturing the plurality of fibroblasts in a medium comprising DA-DKP for a time period sufficient for the plurality of fibroblasts to produce extracellular matrix (ECM).

3. The method of claim 1 or claim 2, wherein the plurality of fibroblasts is cultured in the medium comprising DA-DKP for at least about 2 weeks.

4. The method of claim 2, wherein the plurality of fibroblasts is cultured in the medium comprising serum for at least about 2 weeks.

5. The method of claim 2, wherein steps (b) and (c) are performed sequentially, in any order.

6. The method of claim 2, wherein steps (b) and (c) are performed concurrently.

7. The method of any of the preceding claims, wherein gradually reducing the concentration of serum in the medium over a time period comprises reducing the amount of serum in the medium over the time period such that there is at least a 95% reduction in the concentration of serum.

8. The method of any one of the preceding claims, wherein concentration of serum in the medium is gradually reduced over a time period of at least 5 days.

9. The method of anyone of the preceding claims, wherein the medium comprising serum comprises serum at a concentration of about 0.1% to about 20% (v/v).

10. The method of anyone of the preceding claims, wherein the medium comprising DA-DKP comprises DA-DKP at a concentration of about 1 nM to about 1 μM.

11. The method of anyone of the preceding claims, wherein the medium comprising DA-DKP comprises DA-DKP at a concentration of about 1 μM to about 10 mM.

12. The method of anyone of the preceding claims, wherein the medium comprising DA-DKP comprises DA-DKP at a concentration of about 1 μM to about 1 mM.

13. The method of anyone of the preceding claims, wherein the medium comprising DA-DKP comprises DA-DKP at a concentration of about 100 μM to about 1 mM.

14. The method of anyone of the preceding claims, wherein the plurality of fibroblasts comprises activated fibroblasts.

15. The method of claim 14, wherein the activated fibroblasts express elevated levels of at least one of:
a) Fibroblast Activation Protein (FAP);
b) at least one cytokine, wherein the at least one cytokine is selected from IL-6, IL-8, TGF-β and MIP-1α; and
c) at least one ECM protein, wherein the ECM protein is selected from a laminin, fibronectin, collagen type I, collagen type II, collagen type III, collagen type IV, collagen type V, collagen type VI.

16. The method of any one of the preceding claims, wherein the DA-DKP is obtained from a solution comprising serum albumin.

17. The method of claim 16, wherein the serum albumin is recombinant serum albumin.

18. The method of any one of the preceding claims, wherein the DA-DKP is obtained from conditioned medium that was used to culture a plurality of mammalian cells.

19. The method of any one of the preceding claims, wherein the DA-DKP is obtained from serum.

20. The method of any one of the preceding claims, wherein the DA-DKP was chemically synthesized.

21. The method of any one of the preceding claims, wherein the amount of ECM that is produced is at least about 10%, or at least about 50%, or at least about 100% greater than the amount of ECM that is produced under otherwise identical conditions except for the omission of DA-DKP.

22. The method of any one of the preceding claims, wherein the amount of ECM that is produced is at least about 2 times, or at least about 5 times, or at least about 10 times greater than the amount of ECM that is produced under otherwise identical conditions except for the omission of DA-DKP.

23. The method of any one of the preceding claims, wherein the ECM that is produced comprises soluble ECM, mature ECM, soluble mature ECM or any combination thereof.

24. The method of any one of the preceding claims, wherein the ECM that is produced comprises triple-helical or non-reducible gamma-form fibrillary collagen, or a combination of both.

25. The method of any one of the preceding claims, wherein the ECM that is produced comprises about 90% (w/w/) of COL1, and about 10% (w/w) of COL3, COL4, COL5, COL6, or any combination thereof.

26. The method of any one of the preceding claims, further comprising isolating the ECM.

27. The method of claim 26, wherein the isolated ECM is xeno-free.

28. The method of claim 27, wherein the isolated ECM is substantially free of contaminants which foreign to the human body.

29. A composition comprising the ECM produced by the method of any one of the preceding claims.

30. The composition of claim 29, wherein the composition further comprises DA-DKP.

31. The composition of claim 29 or claim 30, wherein the composition further comprises a plurality of fibroblasts.

32. The composition of any one of claims 29-31 for use as a cosmetic.

33. The use of claim 32, wherein the cosmetic is used for at least one of filling facial wrinkles, reducing visible aging signs, promoting hair growth, improving appearance of skin or any combination thereof.

34. The composition of any one of claims 29-31, for use in the treatment and/or prevention of a disease or disorder.

35. The use of claim 34, wherein the disease or disorder is arthritis, cancer, an autoimmune disorder, a surgical wound, pain or any combination thereof.

36. A kit comprising the ECM produced by the method of any one of claims 1-28.

37. The kit of claim 36, wherein the kit further comprises DA-DKP.

38. The kit of claim 36 or claim 37, wherein the kit further comprises a plurality of fibroblasts.
